SEQUENCE LISTING (5 sequences)

SEQ ID NO: 1 - RNA, 27 nt, Artificial
  Model Common region
cgcagugagggagagaaguacuugcgc

SEQ ID NO: 2 - RNA, 25 nt, Artificial
  Model Specifier region
gcgcaaggaaaggcacuccugagca

SEQ ID NO: 3 - RNA, 17 nt, Bacillus subtilis
cucgccugccacgcggg

SEQ ID NO: 4 - RNA, 27 nt, Bacillus subtilis
ugcagugagagaaagaaguacuugcgu

SEQ ID NO: 5 - RNA, 25 nt, Bacillus subtilis
acgcaacgaaaggcauucuugagca

(19) United States
(12) Patent Application Publication
(10) Pub. No.: US 2008/0026389 A1
(43) Pub. Date: Jan. 31, 2008

(54) SCREEN FOR ANTI-INFECTIVE COMPOUNDS

(76) Inventor: Paul F. Agris, Raleigh, NC (US)

Correspondence Address: MYERS BIGEL SIBLEY & SAJOVEC, PO BOX 37428, RALEIGH, NC, 27627, US

(21) Appl. No.: 11/750,558

(22) Filed: May 18, 2007

Related U.S. Application Data
(60) Provisional application No. 60/832,922, filed on Jul. 24, 2006.

(51) Int. Cl. C12Q 1/68; C07H 21/04

(52) U.S. Cl. 435/6; 536/24.32

(57) ABSTRACT

The present invention provides methods and compositions for identifying compounds useful as therapeutics and disinfectants. Such compounds would be particularly useful in treating Gram-positive bacterial infections. Such therapeutics would target the interaction of a tRNA molecule with an mRNA molecule, interrupting translation of the mRNA molecule and thus interfering with gene expression for a target gene critical to the survival of these organisms.

### RELATED APPLICATIONS

This application claims the benefit of U.S. Provisional Patent Application Ser. No. 60/832,922, filed Jul. 24, 2006, the disclosure of which is incorporated by reference herein in its entirety.

### GOVERNMENT SUPPORT

This work was supported by grants from the National Science Foundation (MCB96-31103 and MCB99-86011) and a grant from the National Institutes of Health (1R01-GM23037).

### FIELD OF THE INVENTION

This invention relates to the field of antibiotic compounds. More specifically the invention provides methods and compositions for identifying compounds useful as therapeutics and disinfectants against bacterial infections.

### BACKGROUND OF THE INVENTION

Bacterial infections pose one of the largest threats to human health. The Gram-positive bacteria are a group of bacteria that include many serious human pathogens such as Staphylococcus, Streptococcus, and Bacillus. These bacteria are increasingly developing multi-drug resistance. Sepsis and other infections by multi-drug resistant bacteria are endemic to hospitals with some strains resistant to even the drug of last resort, vancomycin.

Antibiotic resistance is the ability of a microorganism to withstand the effects of an antibiotic drug. For example, Staphylococcus aureus is one of the major resistant pathogens and was the first bacterium in which penicillin resistance was found in 1947. Methicillin-resistant S. aureus (MRSA) was detected in 1961 and is now found regularly in hospitals. Half of all S. aureus infections in the US are resistant to penicillin, methicillin, tetracycline, and erythromycin. Vancomycin-resistant S. aureus was first identified in 1997 and Linezolid-resistance was reported in 2003. The development of multi-drug resistance is not unique to Staphylococcus. It has also been reported for in other Gram-positive bacteria such as Streptococcus and Enterococcus. It is therefore imperative to find alternate targets of intervention for Gram-positive bacterial infections, ideally ones that are significantly less likely to undergo mutation to resistance.

The T box transcription termination control system is commonly and uniquely used in many Gram-positive bacteria, including pathogenic species, to regulate expression of genes encoding aminoacyl-tRNA synthetases, amino acid biosynthetic enzymes, and transporter proteins (F J Grundy, et al. (2003) Front. Biosci. 8:d20-d31; F J Grundy, et al. (1993) Cell 74:475-482). Each gene in this family is induced in response to a decrease in aminoacylation of the cognate transfer RNA (tRNA). Transfer RNA is a small RNA molecule that transfers a specific amino acid to a growing polypeptide chain at the ribosomal site of protein synthesis during translation. It has a site for amino acid attachment and a three-base region called the anticodon that recognizes the corresponding three-base codon region on messenger RNA (mRNA) via complementary base pairing. Aminoacylation is the process of adding an aminoacyl group to the tRNA molecule, producing tRNA molecules with a covalently linked amino acid. Each tRNA is aminoacylated or “charged” with a specific amino acid by an aminoacyl tRNA synthetase.

Genes in the T box family contain a 5′-untranslated region (5′-UTR) ranging from 200-300 nucleotides (nt) in length having a complex pattern of conserved sequence and structural elements, and generally including an intrinsic transcriptional terminator. Formation of a terminator helix in the nascent mRNA results in premature termination of transcription and repression of transcription of the downstream coding sequence, while sequestration of the 5′ side of the terminator helix into a competing antiterminator structure results in read-through of the termination site and continued transcription (FIG. 1). Folding of the nascent mRNA into the antiterminator structure depends on interaction of the nascent mRNA with the cognate unacylated tRNA. Each gene regulated by this mechanism responds specifically to the cognate tRNA, and specificity is determined by pairing of the anticodon of the tRNA with a “Specifier Sequence” in the “Specifier Loop” of the 5′-UTR. The terminator helix is more stable than the antiterminator, so that preventing termination requires stabilization of the antiterminator. Acylation of the tRNA blocks its interaction with the antiterminator, allowing the system to monitor tRNA charging to signal the requirement for expression of the regulated gene.

The transcription of genes in the T box family is thus regulated through interaction of unacylated tRNA with the 5′-untranslated region (5′-UTR) of the nascent mRNA. Because Gram-positive bacteria regulate gene expression of amino acid metabolic enzymes through this mechanism, and these genes are known to be critical for survival, this regulation mechanism is an ideal target for intervention for Gram-positive bacterial infections and/or disinfectants.

### SUMMARY OF THE INVENTION

In one embodiment the present invention provides a method for identifying a compound that interferes with the binding of a tRNA molecule to a nascent mRNA molecule by contacting the compound with an RNA reporter construct and then measuring the binding of the RNA reporter construct with the tRNA molecule in the presence of the compound.

In another embodiment the present invention provides an RNA reporter construct comprising a reporter molecule and an RNA molecule representing the 5′ UTR of a nascent mRNA molecule of a target gene and useful for measuring the binding of a tRNA molecule to a nascent mRNA molecule.

In another embodiment the present invention provides an anti-infective and/or disinfecting compound for Gram-positive bacteria identified by the method of contacting the compound with an RNA reporter construct and then measuring the binding of the RNA reporter construct with a tRNA molecule in the presence of the compound.

### BRIEF DESCRIPTION OF THE DRAWINGS

FIG. 1 represents the secondary structure of the 5′-UTR of the gram positive, Bacillus subtilis glycyl-tRNA synthetase (glyQS) mRNA. The proposed secondary structure of the leader sequence (nucleotides 1-223) is shown on the left in the Antiterminator form. The Specifier Loop in Stem I contains the glycine codon sequence GGC (nucleotides 99-101) and is responsible for selectively binding the tRNA^{Gly}_{GCC} anticodon GCC in the anticodon stem and loop domain. The Antiterminator bulge (nucleotides 151-161) contains the sequence UGGA that binds the ubiquitous 3′ terminal UCCA sequence of the unacylated tRNA. In the absence of unacylated tRNA, the stem and bulge containing the UGGA sequence takes the alternate conformation of the Terminator (right). Nucleosides with asterisks (*) are conserved among T box mRNAs. The figure is not drawn to scale.

FIG. 2 represents the Specifier Loop region of the glyQS 5′-UTR. [A] Native sequences for the 5′- and 3′-sides of the bottom portion of Stem I. Nucleosides with asterisks (*) are conserved among T box RNAs. Native sequence A-U pairs in red were substituted with G•C base pairs. [B] The bimolecular model RNAs. The Specifier half molecule contains the Specifier Loop with the glycine codon GGC. The sequences were altered to increase the stability of the complex between the Common and Specifier half molecules, including the addition of a predicted base pair between C₃₅:G₉₄ denoted with open circles (O). The Specifier half molecule was synthesized with the reporter 2-aminopurine (2AP) substituting for A either 5′- or 3′ to the codon, at position 98 or position 102 (circled A). The 2AP (inset) fluorescence was used to monitor interactions. The 17 nt ASL^{Gly}_{GCC} is shown in the secondary structure determined from NMR analyses.

FIG. 3 represents reconstitution of Stem I from interaction of the Specifier and Common half molecules. [A] Formation of the Specifier/Common complex was observed by PAGE mobility shift analyses. Specifier 2AP₉₈ half molecule (183 μM) was titrated with increasing concentrations of the Common half molecule (lane 2, 0.0; 3, 23.9; 4, 47.9; 5, 71.8; 6, 95.8; 7, 120; 8, 144; 9, 287 μM). RNA standards, a 17-mer anticodon stem and loop and unfractionated tRNA ˜76mer were run in lanes 1 and 10. In addition, lane 1 contained Specifier half molecule (25mer), and lane 10 contained Common half molecule (27-mer). [B] Complex formation between the Common half molecule and the two Specifier half molecules, 2AP₉₈ (squares; ▪) and 2AP₁₀₂ (circles; ●). Ethidium bromide stained RNAs of the PAGE mobility shift analyses were quantified with ImageQuant, the results normalized and plotted as percent Complex (bound) vs. concentration of the Common half molecule. Binding curves were analyzed with a non-linear regression (Origin) to extrapolate dissociation constants. The error for multiple determinations of the K_{d}s was determined be ±1.0 to ±2.3 μM.

FIG. 4 represents fluorescence spectroscopy of binding of the Specifier 2AP₉₈ to Common and to ASL^{Gly}_{GCC}. [A] Fluorescence emission spectra were recorded for Specifier 2AP₉₈ alone [diamonds; ♦], reconstitution of the 5′-UTR Stem I complex between Specifier 2AP₉₈ and Common [squares; ▪], the binding of ASL^{Gly}_{GCC} to the Specifier 2AP₉₈ and Common complex [diamonds; ♦], and the non-binding control of ASL^{Phe}_{GAA} [triangles; ▴]. Spectra in the presence of the ASLs have unexplained shoulders in the 400-450 nm range. [B] Binding of the Specifier 2AP₉₈ half molecule to the Common half molecule. With the Specifier 2AP₉₈ concentration constant (2.0 μM), the concentration of the Common half molecule was increased. Data were normalized and the percent fluorescence quenching (bound) plotted vs. Common concentration. The binding curves were analyzed with a non-linear regression (Origin). The error (±0.7 μM) was derived from the average of three experiments (squares, circles, triangles; ▪, ●, ▴). [C] Binding of ASL^{Gly}_{GCC} to the Specifier 2AP₉₈/Common complex. The concentration of the Specifier 2AP₉₈/Common complex was held constant (2.0 μM) as ASL^{Gly}_{GCC} was titrated into solution. Data were normalized and the percent fluorescence quenching (bound) plotted vs. ASL^{Gly}_{GCC} concentration. The binding curves were analyzed with a non-linear regression (Origin). The dissociation constant was derived from the average of two runs of the experiment (squares, circles; ▪, ●).

FIG. 5 represents fluorescence spectroscopy of binding of the Specifier 2AP₉₈ with the valine codon to ASL^{Val}_{UAC}. Fluorescence emission spectra were recorded for the Val Specifier 2AP₉₈ alone [diamonds; ♦], reconstitution of the 5′-UTR Stem I complex between Val Specifier 2AP₉₈ and Common [squares; ▪], the binding of ASL^{Val}_{UAC} to the Val Specifier 2AP₉₈ and Common complex [diamonds; ♦], and the non-binding control of ASL^{Arg}_{CCG} [triangles; ▴].

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

As used herein, “a,” “an” or “the” can mean one or more than one. For example, “a” cell can mean a single cell or a multiplicity of cells.

As used herein, the term “RNA molecule” refers to a molecule comprising ribonucleic acid (RNA). The RNA molecule may represent a fragment of a nascent mRNA molecule for a target gene. In a preferred embodiment, the RNA molecule represents the 5′ UTR of a nascent mRNA molecule of a target gene. Methods for making RNA molecules are known to those skilled in the art and include chemical synthesis of synthetic RNA.

An “mRNA molecule” or “messenger RNA molecule” is an RNA molecule produced through the process of transcription from a DNA molecule. The sequence of an mRNA molecule contains a complimentary copy of the gene's protein-coding DNA sequence as well as other sequences such as 5′ and 3′ untranslated regions. An mRNA molecule may be used to produce a protein through the process of translation.

A “nascent mRNA” or “nascent mRNA molecule” is an mRNA molecule that is newly formed or in the process of being formed. A nascent mRNA molecule is formed during the process of transcription. A nascent mRNA molecule may or may not be destined to become a fully formed mRNA molecule.

A “tRNA” or “tRNA molecule” is generally an RNA molecule that transfers a specific amino acid to a growing polypeptide chain at the ribosomal site of protein synthesis during translation of an mRNA molecule. Each tRNA molecule is specific for the type of amino acid it transfers and the mRNA codon sequence it binds (with its anticodon). The tRNA recognizes the corresponding three-base codon region on mRNA via complementary base pairing. In one embodiment, the tRNA molecule used in practicing the present invention is a naturally occurring tRNA molecule. In another embodiment, the tRNA molecule used in practicing the present invention is not a naturally occurring tRNA molecule, or is a fragment of a natural tRNA molecule, that retain the anticodon (e.g., a fragment at least 4, 6, 8, 10, 12, or 16 nucleotides in length). Such tRNA molecule may be modified or designed for use in an in vitro screen and may not necessarily represent a naturally occurring tRNA molecule and may not be functional in vivo.

As used herein, the term “nucleic acid sequence” or “sequence” refers to the sequence of nucleotides from the 5′ to 3′ end of nucleic acid molecule. Nucleic acid sequences provided herein are represented using the standard code for representing the nucleotide characters as set forth in the U.S. sequence rules, 37 CFR §§1.821-1.825 and the World Intellectual Property Organization (WIPO) Standard ST.25.

A “5′ untranslated region” or “5′ UTR” is a section of a gene located before the start codon. This region is transcribed from the DNA molecule as part of the same mRNA molecule as the coding region. The 5′ UTR sequence in the mRNA molecule is complementary to the gene's DNA sequence but is not translated directly into a protein sequence. The 5′ UTR may contain specific sequences that affect transcription and/or translation of the mRNA sequence. Several roles in gene expression have been attributed to the untranslated regions, including mRNA stability, mRNA localization, and translational efficiency. The ability of a UTR to perform these functions depends on the sequence of the UTR and can differ between mRNA molecules and genes.

As used herein, the term “gene” refers to a nucleic acid molecule capable of being used to produce mRNA or antisense RNA. Target genes of the present invention include any gene critical for the survival of an organism. Such target genes include a gene having a 5′ UTR transcription and/or translation control system and encoding a metabolic enzyme. An exemplary target gene would be a gene having a T box transcription termination control system and encoding an amino acid metabolic enzyme. Examples of these amino acid metabolic enzymes are the aminoacyl-tRNA synthetases, amino acid biosynthetic enzymes, and amino acid transporter proteins.

A T box transcription termination control system is a system for regulating the expression of genes having a T box in their 5′ UTR. The system is widely used for control of gene expression in Gram-positive bacteria, but is rare in Gram-negative organisms. The T box system regulates expression of specific genes in Gram-positive bacteria through premature termination of transcription. The system relies on the binding of a cognate uncharged tRNA molecule to a sequence in a nascent mRNA molecule to stabilize an antiterminator element and thus allow for the synthesis of the full-length mRNA. Methods for identification of T box transcription termination control system sequences and genes containing these are known in the art (See e.g., F J Grundy, et al. (2003) Frontiers in Bioscience 8:d20-31, all of which is herein incorporated by reference). Other 5′ UTR transcription and/or translation control systems would also be useful in practicing the present invention.

As used herein, the term “Gram-positive bacteria” includes those bacteria that are stained dark blue or violet by Gram staining such as Bacillus, Listeria, Staphylococcus, Enterococcus, Lactobacillus, Lactococcus, Leuconostoc, Pectinatus, Pediococcusm, Streptococcus, Acetobacterium, Clostridium, Eubacterium, Heliobacterium, Heliospirillum, Sporomusa, and Actinobacteria. The Gram-positive bacteria also includes bacteria that lack cell walls and so cannot be stained by Gram but are nonetheless related to bacteria that can be stained by Gram. Examples of these include but are not limited to Mycoplasma, Spiroplasma, Ureaplasma, and Erysipelothrix. The present invention is not to be construed, however, as limited to only genes from Gram-positive bacteria.

In one embodiment, the invention provides a method for screening a compound for antibiotic activity and/or disinfecting activity. The activity would be through interference by the compound with the control of gene expression in gram-positive bacteria. In furtherance of this, in one embodiment the screen targets the T box transcription termination control system (AR Nelson, et al. (2006) RNA, 12(7):1-8, all of which is herein incorporated by reference). The screen does not require any proteins, can be done in vitro, and targets an RNA/RNA interaction. The screen takes advantage of small molecule chemistry that selectively recognizes:

a) the unique combination of chemistry and structure of the Specifier loop or segment presentation of codon and 3′-adjacent purine;

b) a possibly unique four-base pair interaction of codon with anticodon (plus one additional base); and

c) possible modification chemistries known to be required of some tRNAs to bind codon on the ribosome, and possible unique to the pathogen, and thus not in the human host.

Because of this, the tRNA anticodon recognition of the 5′ UTR of the nascent mRNA molecule for a gene is likely to be species specific for some genes. In addition, this is an ideal target for antibiotics since the organism would find it difficult to circumvent this interaction through mutation because it is very similar to the interaction of tRNA with mRNA codon in the A-site of the ribosome.

The three base anticodon of each regulating tRNA interacts with a three base codon sequence (the “Specifier”) in the 5′ UTR of the nascent mRNA molecule though complementary base pairing in the absence of protein and potentially with a fourth base pair, distinguishing the interaction from that on the ribosome. An experimental model of this interaction has been synthesized and tested and is described further herein. The 5′ UTR of a nascent mRNA molecule presents the codon and selectively binds the specific anticodon of a tRNA molecule. This interaction can be observed in the experimental model by fluorescence quenching of a fluorescent reporter molecule located adjacent to the codon. Fluorescent quenching is specific to the proper codon/anticodon interaction. Changes in fluorescence are readily observed with a fluorescence reader and can be measured in a high throughput manner using a microplate format. Large numbers of candidate therapeutics could thus be screened in this manner.

Compounds to be Screened. As used herein, the term “compound” encompasses any chemical compound or molecule, including but not limited to small molecules, proteins, peptides (e.g., peptides of from 3 or 5 to 50 or 100 amino acids in length, or more), antibodies, nucleosides, nucleotides, oligonucleotides, modified nucleosides, modified nucleotides, modified oligonucleotides, etc. Thus a variety of agents from various sources can be screened for their abilities to inhibit the control of gene expression by tRNA by using the methods of the present invention. Agents to be screened can be naturally occurring or synthetic molecules. Agents to be screened can also obtained from natural sources, such as, e.g., marine microorganisms, algae, plants, fungi, etc. Alternatively, agent to be screened can be from combinatorial libraries of agents, including peptides or small molecules, or from existing repertories of chemical compounds synthesized in industry, e.g., by the chemical, pharmaceutical, environmental, agricultural, marine, cosmeceutical, drug, and biotechnological industries. Agents can include, e.g., pharmaceuticals, therapeutics, environmental, agricultural, or industrial agents, pollutants, cosmeceuticals, drugs, organic compounds, lipids, glucocorticoids, antibiotics, peptides, proteins, sugars, carbohydrates, chimeric molecules, etc. (see, e.g., U.S. Pat. No. 7,041,276). RNAs that can be screened include but are not limited to synthetic and naturally occurring RNAs, including but not limited to short hairpin RNAs (shRNAs), examples of which include noncoding regulatory RNAs (ncRNAs) such as small interfering RNA (siRNA), micr RNA (miRNA), small nuclear RNA (snRNA), small non-mRNA (snmRNA), small nucleolar RNA (snoRNA), small temporal RNA (stRNA) etc. See, e.g., PCT Application WO 2005/102298. Antibodies to be screened include any natural or synthetic antibodies, including immunoglobulins such as IgG, IgM, IgA, IgD, and IgE. The antibodies may be monoclonal or polyclonal and may be of any species of origin, including (for example) mouse, rat, rabbit, horse, or human, or may be chimeric antibodies. See, e.g., M. Walker et al., Molec. Immunol. 26, 403-11 (1989). The antibodies may be recombinant monoclonal antibodies produced according to the methods disclosed in Reading U.S. Pat. No. 4,474,893, or Cabilly et al., U.S. Pat. No. 4,816,567. All compounds to be screened may be combined, conjugated, coupled to or complexed with additional carrier or delivery compounds (e.g., proteins, peptides or any compound described in connection with compounds to be screened above) for presentation or delivery.

The compound to be screened can be a member of a compound library. Compound libraries or combinatorial libraries can be produced for many types of compounds that can be synthesized in a step-by-step fashion. Such compounds include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of compounds can be constructed by the encoded synthetic libraries (ESL) method described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety for all purposes). Peptide libraries can also be generated by phage display methods. See, e.g., Devlin, WO 91/18980. Compounds to be screened can also be obtained from governmental or private sources, including, e.g., the National Cancer Institute's (NCI) Natural Product Repository, Bethesda, Md., the NCI Open Synthetic Compound Collection, Bethesda, Md., NCI's Developmental Therapeutics Program, or the like (see, e.g., U.S. Pat. No. 7,041,276).

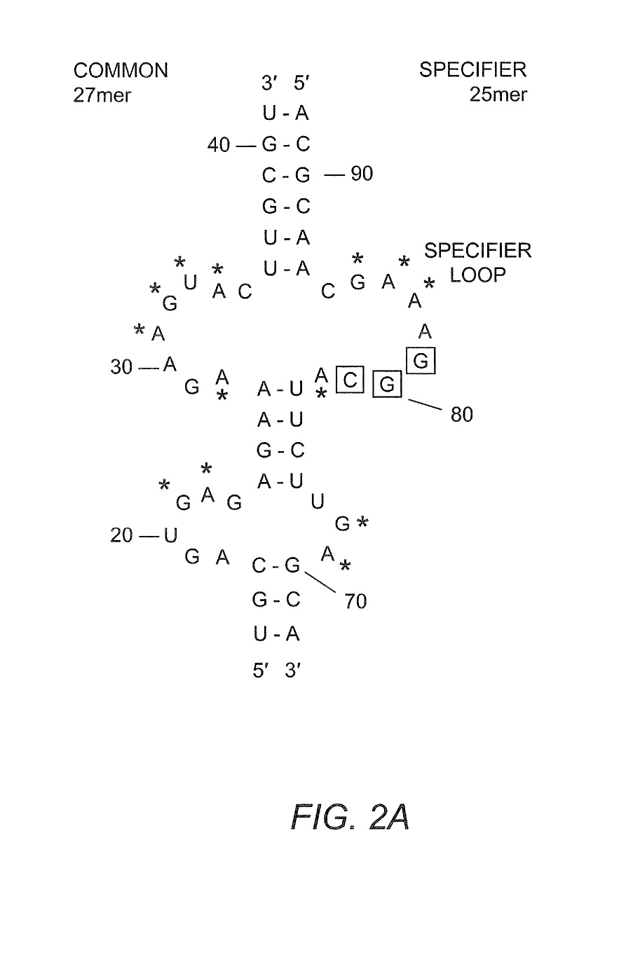

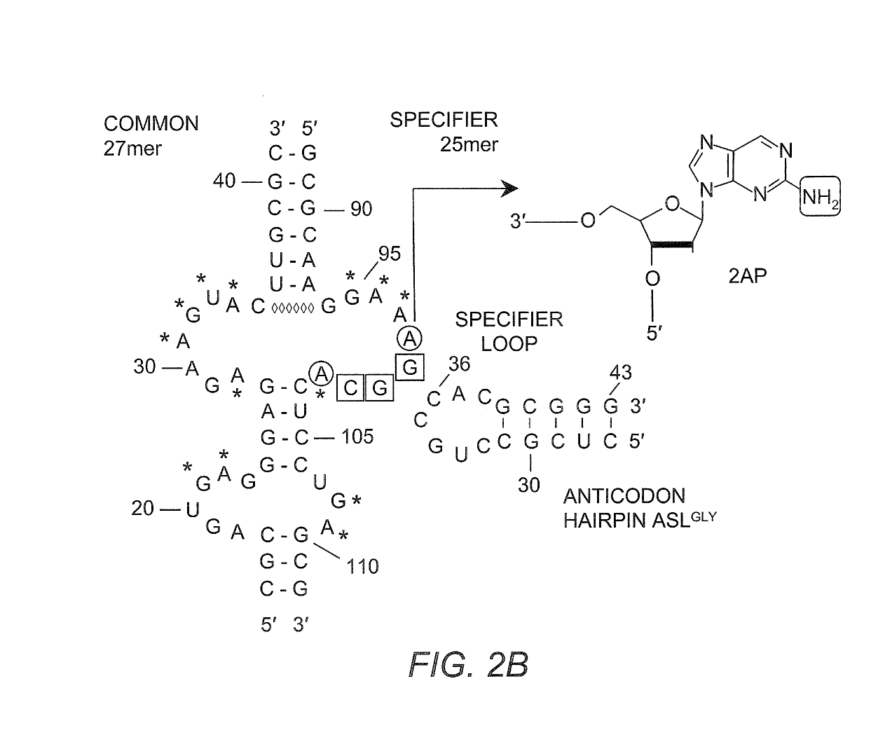

In another embodiment, the invention provides an RNA reporter construct for measuring the binding of a tRNA molecule to an RNA molecule. As used herein, the term “RNA reporter construct” or “construct” refers to a construct capable of binding to a complimentary tRNA molecule and comprising a reporter molecule that can be used to measure or indicate such binding. The construct thus comprises (1) an RNA molecule representing the 5′ UTR of a nascent mRNA molecule of a target gene and (2) a reporter molecule. Exemplary 5′ UTR's are those that contain a T box transcription termination control system. The RNA molecule comprises a Specifier Loop comprising a Specifier codon that is complimentary to the anticodon of a tRNA molecule. See, for example, FIG. 2 herein. The construct can be unimolecular (i.e. one molecule, such as when stem 1 is included in the construct) or can comprise more than one molecule (i.e. at least bimolecular, such as when stem 1 is excluded from the construct, and/or when a common oligonucleotide such as the common 27mer is hybridized to the specific oligonucleotide by Watson-Crick pairing). A bimolecular construct allows for the Specifier codon or segment to be changed without re-synthesizing the entire RNA molecule. In either case, an advantage of the present invention is the shortening of the construct, such that the RNA molecule representing the 5′ UTR need only be up to 50, 100, or 150 nucleotides in length (e.g., where a single oligonucleotide containing a stem is employed), or need only be up to 40, 50 or 60 nucleotides in length (e.g., where a bimolecular construct is employed in which the RNA molecule is hybridized to a common oligonucleotide). In general, in both situations, the RNA molecule representing the 5′ UTR is preferably at least 20 or 22 nucleotides in length.

As used herein, the term “reporter molecule” refers to any molecule capable of being used to measure a specific result. Preferred reporter molecules of the present invention are ones that can be measured visually, such as fluorescent reporter molecules like 2-aminopurine ribonucleoside (2AP). The reporter molecule of the RNA reporter construct of the present invention is ideally located at a position that is sensitive to codon/anticodon interaction but does not interfere with the presentation of the codon and the interaction.

### EXAMPLES

An assay for screening a compound to determine whether it interferes with the binding of a tRNA to a nascent Bacillus subtilis glyQS RNA molecule was designed and tested. The assay comprised a bimolecular RNA construct in which the interaction between two half molecules (“Common” and “Specifier”) would reconstitute the Specifier Loop region of the 5′-UTR of the nascent mRNA for the Bacillus subtilis glyQS gene. The reconstituted 5′-UTR of the glyQS nascent mRNA was tested with the anticodon stem and loop of tRNA^{Gly} (ASL^{Gly}_{GCC}). The bimolecular RNA construct when tested with ASL^{Gly}_{GCC} was found to mimic the RNA-RNA interaction required for T box gene regulation in vivo.

Design of a Bimolecular System to Mimic the T Box Specifier Loop Domain. The structural arrangement of the bottom half of the B. subtilis glyQS 5′-UTR Stem I domain was generated from RNAs corresponding to the 5′ (“Common,” 27 nt) and 3′ (“Specifier,” 25 nt) regions. The Common half of this bimolecular complex corresponds to glyQS residues 15-41, while the Specifier half corresponds to residues 88-112 (FIG. 2A). Residues conserved in T box family sequences were maintained in the model sequences, while certain A-U pairings within helical domains were replaced with G-C pairings at positions where G-C pairings are found in other T box sequences. Specifier half molecule constructs were synthesized with or without a fluorescence reporter in the form of a single 2-aminopurine ribonucleoside (2AP) substitution at A₉₈ or A₁₀₂, on either side of the GGC glycine codon (FIG. 2B). Neither A₉₈ nor A₁₀₂ are invariant residues, although the position corresponding to A₁₀₂ is conserved as a purine and pairs with the conserved U33 residue of tRNA. A 17 nt RNA hairpin structure, comprising residues 27-43 of tRNA^{Gly}_{GCC} was synthesized and used as a mimic of the anticodon stem and loop domain (FIG. 2B).

RNA Synthesis. RNAs corresponding to the 5′ (“Common”) and 3′ (“Specifier”) sequences of the region at the base of the Stem I element of the B. subtilis glyQS 5′-UTR (FIG. 2A) were chemically synthesized. Two Common half molecules were synthesized. The wild type sequence was comprised of the most highly conserved nucleosides and sequences (FIG. 2B). A mutant Common sequence was synthesized with an altered K-turn motif in which A₂₂ was substituted with G₂₂. The Specifier Loop contained either the glycine codon GGC or the valine codon GUA (Gly Specifier and Val Specifier, respectively). To increase the stability of helical elements, particular U-A or A-U base pairs were selected and replaced with C-G or G-C pairs (FIG. 2B). In addition, C94 was replaced with a G to potentially add an extra base-pair in the helix above the Specifier Loop (FIG. 2B). These changes represent natural variations found in other T Box RNA sequences (unpublished results). The Specifier half molecule (FIG. 2B) was synthesized with and without 2-aminopurine ribonucleoside (2AP) at positions A₉₈ or A₁₀₂, flanking the codon sequence. A 17 nt RNA (ASL^{Gly}_{GCC}) was designed and chemically synthesized with the anticodon GCC to mimic the anticodon stem and loop domain of B. subtilis tRNA^{Gly}_{GCC} (FIG. 2B). An ASL^{Val}_{UAC} was synthesized to test binding to the Val Specifier. Two negative control ASLs also were chemically synthesized, an ASL^{Phe}_{GAA} with the GAA phenylalanine anticodon, and an ASL^{Arg}_{CCG} with the arginine anticodon CCG. RNAs were synthesized by ‘ACE’ chemistries (Dharmacon RNA Technologies, Colorado). The RNAs were deprotected as suggested by the manufacturer, lyophilized, and dissolved in H₂O. The purity of the RNA was analyzed by ion exchange HPLC and denaturing (7 M urea) polyacrylamide gel electrophoresis (PAGE).

RNA Folding Predictions. The most thermodynamically stable structures for the Common and Specifier half molecules (individually and in combination) and the ASL RNAs were predicted by using RNAStructure 3.0 (See e.g., D H Mathews, et al. (1999) J. Mol. Biol. 288:911-940; D H Mathews, et al. (2004) Proc. Natl. Acad. Sci. USA. 101:7287-7292).

Polyacrylamide Gel Electrophoresis (PAGE) Mobility Shift Analysis Methods. Binding of the Common and Specifier half molecules was assessed using a PAGE mobility shift assay. The gel composition was 15% polyacrylamide in TB buffer (89 mM Tris base, 89 mM boric acid, pH 8.3). PAGE was conducted at 4±0.5° C. using a temperature controlled gel electrophoresis apparatus (Novex Mini-cell Thermoflow, Invitrogen, Carlsbad Calif.). Concentrations of the Specifier half molecule were kept constant and titrated with increasing amounts of the Common half molecule. The reaction mixtures were heated at 90° C. for 30 min and then allowed to slow cool to room temperature for 45 min before electrophoresis. Prior to loading, the RNA samples were diluted 4-fold (vol/vol) with a loading buffer (TB buffer that was 50% glycerol, 0.25% [w/v] Bromophenol Blue and 0.25% [w/v] Xylene Cyanol FF). After electrophoresis, gels were stained with ethidium bromide (0.5 μg/ml, 15 min, at room temperature) and digitally photographed (BioRad, Calif.). The RNA bands were quantified using ImageQuant software (Molecular Dynamics, Amersham Biosciences, N.J.). Complex formation was normalized to 100% for the lane in which the Specifier RNA had been titrated completely into the complex with the Common RNA. Dissociation constants (K_{d}) were determined using a sigmoidal model (Origin, MicroCal, LLC, Northampton, Mass.).

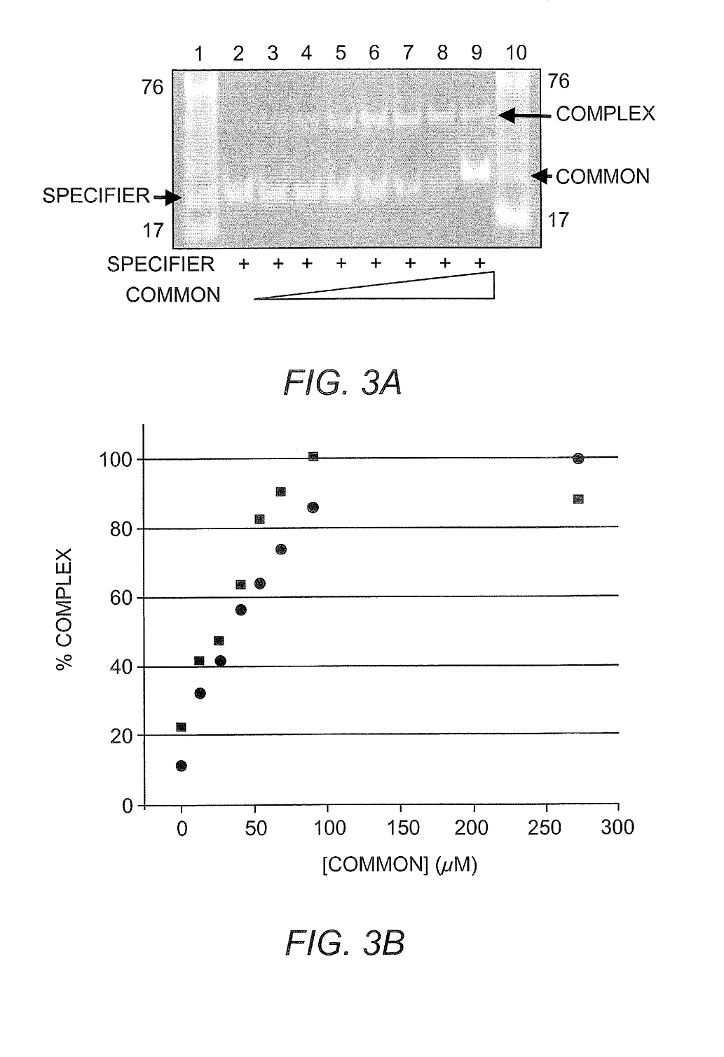

Monitoring the Reconstitution of the Stem I Complex. Formation of a complex between the Common and Specifier half molecules was assessed initially by PAGE mobility shift assays. Addition of increasing concentrations of the Common RNA to a constant amount of the Specifier 2AP₉₈-RNA resulted in appearance of a band with slower migration, the intensity of which increased as higher concentrations of the Common half molecule were added (FIG. 3). The migration of the Specifier/Common complex was consistent with that of a unimolecular RNA of equal size (52mer), as determined from the 17mer and 76mer standards, and individual Specifier (25mer) and Common (27mer) species in the gel. The individual Specifier and Common half molecules did not exhibit slower moving bands even at excessive (300 μM) concentrations. Quantification of the ethidium bromide stained bands yielded binding curves for the interactions of the Common half molecule with Specifier 2AP₉₈ and 2AP₁₀₂ half molecules (FIG. 3B). Dissociation constants (K_{d}s) and standard free energies of binding at equilibrium (ΔG°s) were obtained from the binding curves. The reconstitution of Stem I through the interaction of the Specifier half molecule with the Common half molecule was found to occur with K_{d}s of 27.6±1.0 μM and 32.3±2.3 μM for the Specifier 2AP₉₈ and 2AP₁₀₂ molecules, respectively. The ΔG°s of complex formation from the gel mobility shift assay were 6.2±0.1 Kcal/mol for Specifier 2AP₉₈ and −6.1±0.1 Kcal/mol for 2AP₁₀₂.

Fluorescence Spectroscopy Methods. The fluorescence of 2AP in the Specifier half molecule was observed in order to monitor interactions with the Common half molecule and with the anticodon stem and loop (ASL). Spectra and end point fluorescence data were collected with a microplate reader with two monochrometers (SprectraMax Gemini XS, Molecular Devices, Sunnyvale, Calif.). Excitation and emission wavelengths of 2AP in the Specifier half molecule were 310 nm and 375 nm, respectively. Optimum sensitivity and response for the instrument was found to occur at a concentration of 2 μM for Specifier 2AP₉₈, and at 3 μM for Specifier 2AP₁₀₂. Specifier half molecules were titrated with increasing concentrations of the Common half molecule from 2 to 30 μM. The mixture of Specifier and Common half molecules was denatured at 90° C. for 30 min and then the two half molecules were allowed to anneal at room temperature for 45 min prior to measurement of fluorescence. ASL RNAs were incubated with the already formed complex of Specifier 2AP₉₈/Common at 25° C. for 5 min. The interactions of ASL^{Gly}_{GCC} and ASL^{Phe}_{GAA} with the complex were monitored by collecting fluorescence emission spectra. All of the obtained spectral data were then analyzed in Microsoft Excel and Origin to compare the change in fluorescence among the different complexes. The data were normalized to 100% for the sample exhibiting the most quenching of the fluorescent reporter, indicating the most complex formation. The molar standard Gibbs free energy change (ΔG°) of the Specifier/Common folding interaction, and that of the binding of ASL to the Specifier 2AP₉₈/Common complex were derived from ΔG°=−RTlnK_{eq}, where T=298° K, and

$${K}_{cq} = \frac{[ Specifier / Commoncomplex ]}{[ Specifier ] [ Common ]}$$

$$and$$

$$\frac{[ Specifier / Common / ASL ]}{[ Specifier / Commoncomplex ] [ ASL ]}$$

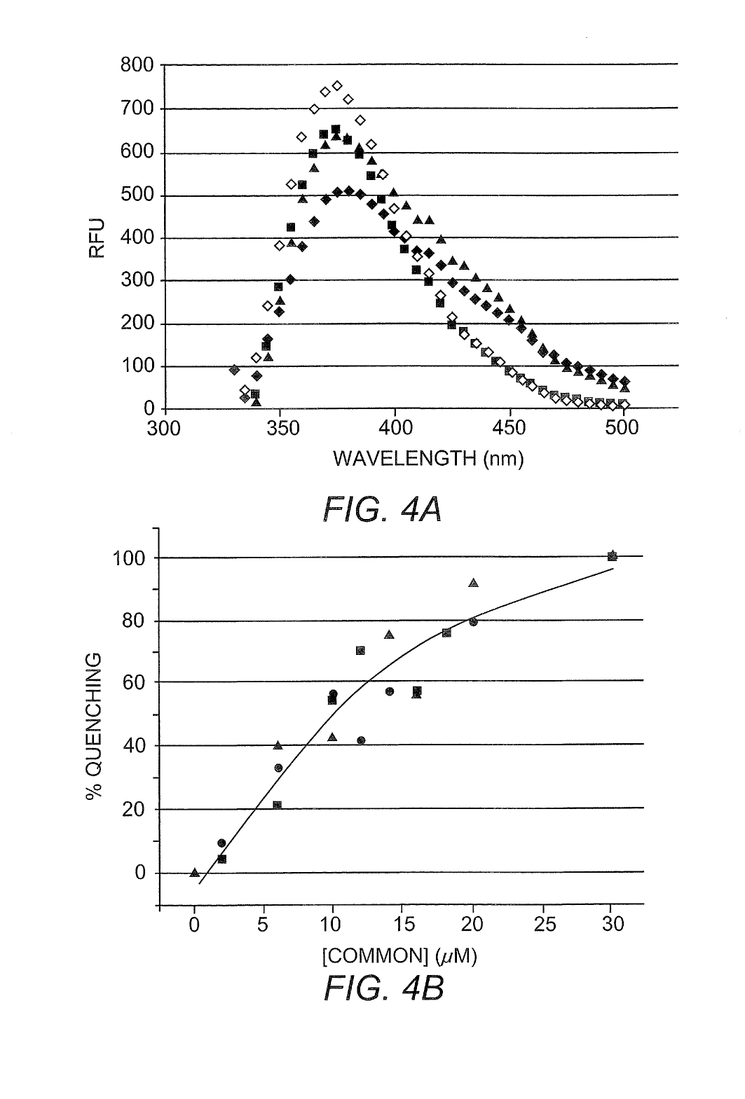

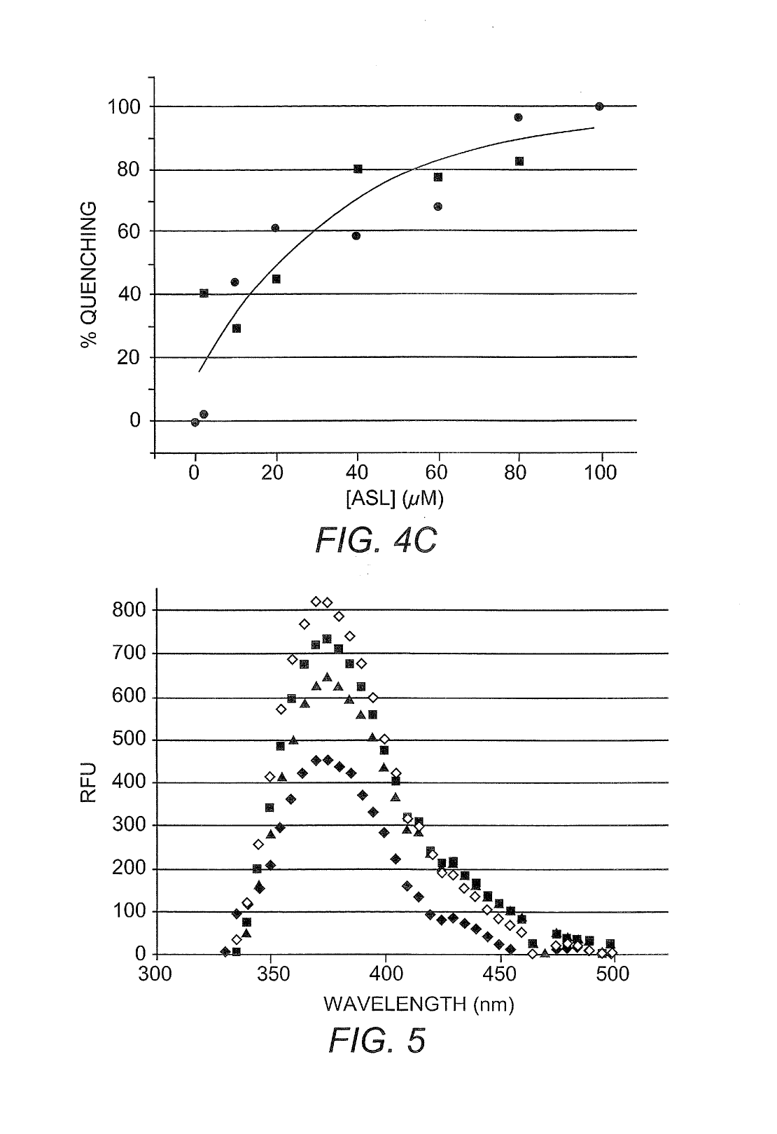

Monitoring of Specifier/Common Complex Formation. Fluorescence of the 2AP₉₈ and 2AP₁₀₂ Specifier half molecules was monitored after incubation with the Common half molecule. Formation of the complex with the Common half molecule quenched the fluorescence of both Specifier RNAs, but did not alter the fluorescence spectral profile (FIG. 4A). The Specifier 2AP₉₈ half molecule was a more sensitive reporter of complex formation than was the Specifier 2AP₁₀₂. In addition, the pairing of A₁₀₂ with the invariant U₃₃ of the tRNA was predicted to contribute to the strength of the codon/anticodon interaction that we wanted to analyze within our experimental model. Therefore, Specifier 2AP₉₈ was used to confirm the binding constant observed by the PAGE mobility shift assay. Titration of Specifier 2AP₉₈ with the Common half molecule resulted in maximal quenching of the fluorescence at 20 μM Common. The affinity of Specifier 2AP₉₈ for the Common half molecule was derived from the binding curves (FIG. 4B) to be 10.5±0.7 μM with a ΔG° of −6.8±0.1 Kcal/mol, almost three-fold stronger than that determined by PAGE mobility shift analyses (27.6±1.0 μM, −6.2±0.1 Kcal/mol). Thermodynamic and free energy calculations to predict the folding interactions of the Specifier and Common half molecules in forming Stem I resulted in two closely related and almost equally stable structures. Both structures displayed the codon within the Specifier loop. However, the second most stable structure with a free energy (ΔG° at 298° K) of −19.2 Kcal/mole most closely resembled the conformation supported by genetic analysis in vivo and chemical and enzymatic probing experiments in vitro. The other structure was only modestly more stable with a ΔG° of −21.0 Kcal/mole.

Monitoring of the Interaction of the Specifier Sequence with the ASL Anticodon. The addition of ASL^{Gly}_{GCC} to the complex formed from the Specifier 2AP₉₈ and Common half molecules led to further quenching of the fluorescence reporter, and alteration of the fluorescence emission spectrum profile (FIG. 4A). Fluorescence shoulders in the spectrum appeared at 420 and 450 nm (FIG. 4A). The Specifier 2AP₉₈/Common complex was titrated with ASL^{Gly}_{GCC} in order to determine the affinity for the ASL anticodon. Fluorescence of the Specifier 2AP₉₈ (2 μM) was not affected by increasing concentrations of ASL^{Gly}_{GCC} beyond 100 μM (FIG. 4C). The affinity of the Specifier 2AP₉₈/Common complex for the ASL^{Gly}_{GCC} was 20.2±1.4 μM and yielded a ΔG° of −6.4±0.1 Kcal/mole. In order to assess the specificity of the interaction, we synthesized an anticodon stem and loop corresponding to that of yeast tRNA^{Phe} with the codon GAA (ASL^{Phe}_{GAA}). In contrast to ASL^{Gly}_{GCC}, addition of the negative control ASL^{Phe}_{GAA} to the Specifier 2AP₉₈/Common complex did not quench the fluorescence (FIG. 4A), even at a concentration (100 μM) at which maximum quenching was achieved by ASL^{Gly}_{GCC}. However, the same two shoulders in the spectrum were observed. Thus, we conclude that the fluorescence shoulders at 420 and 450 nm were not the result of specific interactions between the Specifier Sequence and the ASL anticodon.

Altering the codon in the Specifier loop should change the specificity for tRNA. A Specifier loop was constructed with the GUA codon for valine tRNA and 2AP at position 98 as a reporter of the interaction with the anticodon. When Stem I was reconstituted from the Val Specifier and Common half molecules, fluorescence quenching of 2AP₉₈ was comparable to that with the Gly Specifier (FIGS. 4 and 5). The reconstituted Stem I was bound by ASL^{Val}_{UAC} as evidenced by the significant quenching of the 2AP₉₈ fluorescence. The degree of fluorescence quenching with ASL^{Val} at 100 μM was comparable to the maximum binding achieved with 100 μM ASL^{Gly} to the Gly Specifier (FIG. 5). However, the fluorescence of the Val Specifier 2AP₉₈ was hardly altered with the addition of a negative control, ASL^{Arg}_{CCG} at 100 μM (FIG. 5).

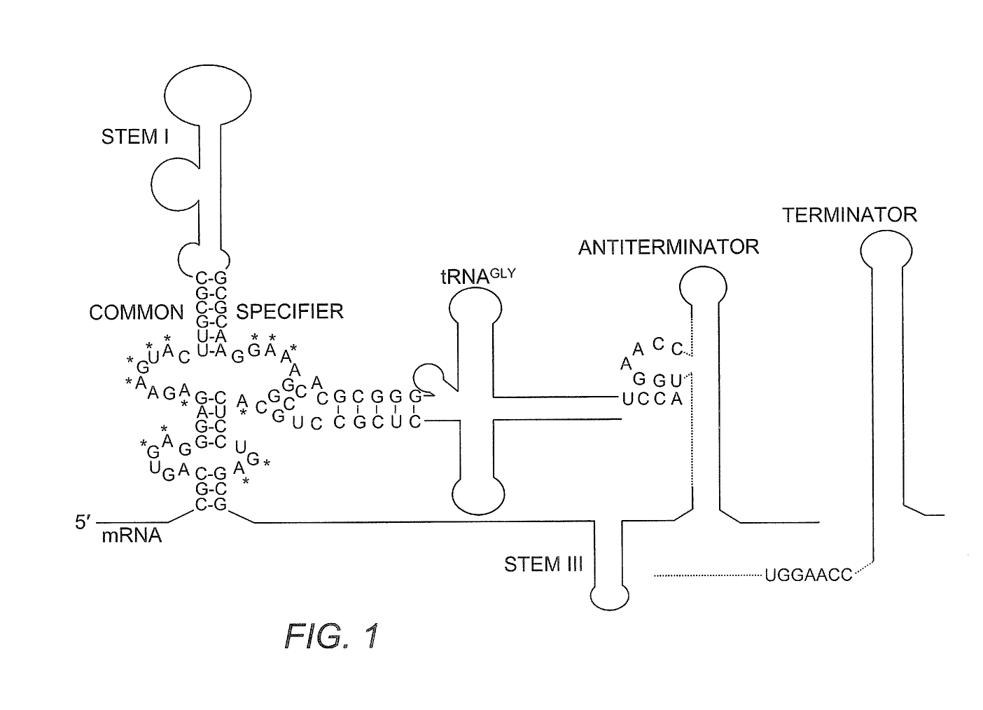

Discussion. This model system was designed for analyzing the physicochemical properties required in the functional folding of the 5′-UTR of GlyRS mRNA and in its binding to tRNA_{Gly}. A model of the 5′-UTR was designed and synthesized as two RNAs, Specifier and Common, to form the bottom portion of the Stem I element of the mRNA leader sequence (FIG. 1), which interacts with the tRNA anticodon region. Formation of the complex was assessed by PAGE and was observable and measurable by staining with ethidium bromide. The ΔG° of complex formation was determined to be −6.2±0.1 Kcal/mole. A gel mobility shift assay monitored by ³²P-end labeling of the Specifier half molecule also detected complex formation and produced a similar free energy of folding, −6.9 Kcal/mole. Mung bean ribonuclease digestion of the RNA indicated that the codon was accessible in the complex. Fluorescence spectroscopy was utilized as a second approach to observing complex formation. The fluorescence of 2AP substituted for adenosine at either side of the Specifier codon was quenched upon interaction of the Specifier half molecule with the Common half molecule. The free energy of complex formation from the fluorescence derived binding constant (−6.8±0.1 Kcal/mole) was comparable to that obtained by gel mobility shift assays. Thus, the empirically derived AGs were comparable to each other, and considerably higher than the −19.2 Kcal/mole of the theoretical folding derived from empirical studies often conducted in the presence of 1 M NaCl (DH Mathews, et al. (1999) J. Mol. Biol. 288:911-940; DH Mathews, et al. (2004) Proc. Natl. Acad. Sci. USA. 101:7287-7292). This difference is reasonable considering that the dynamics of the system in solution assessed by gel mobility shift and by fluorescence spectroscopy and the lower counter ion concentration probably contribute to the higher free energy than that predicted by a static interaction calculation.

Neither the gel mobility shift nor the fluorescence analyses indicate if the reconstituted Stem I structure was providing a stably open Specifier Loop with the codon presented for anticodon binding, properties that are crucial to the biological relevance of the model. To investigate the interaction of the Specifier glycine codon, GGC, with the tRNA^{Gly}_{GCC} anticodon, we designed and synthesized the 17 nt anticodon stem and loop domain ASL^{Gly}_{GCC}. Observation of a specific interaction between the Specifier Sequence and the ASL^{Gly}_{GCC} would contribute indirect proof that the codon was accessible for binding. Addition of ASL^{Gly}_{GCC} to the reconstituted Stem I complex formed from Specifier 2AP₉₈ and Common half molecules quenched the 2AP fluorescence indicating an interaction. The absence of quenching of the 2AP₉₈ fluorescence with the negative control anticodon stem and loop of yeast tRNA^{Phe} (ASL^{Phe}_{GAA}) at 100 μM indicated that the interaction with ASL^{Gly}_{GCC} was specific. The Specifier 2AP₉₈/Common complex bound the ASL^{Gly}_{GCC} RNA with a K_{d} of 20.2±0.1 μM, ΔG°=−6.4±0.1 Kcal/mole. These results indicate that Specifier Loop sequence of the Stem I model RNA was physically and chemically accessible for anticodon binding and bound the ASL^{Gly}_{GCC} specifically. The K_{d} of 20.2±0.1 μM was surprisingly strong considering that the Common/Specifier interaction resulted in a K_{d} of 10.5±0.7 μM. The unassisted interaction of a codon with an anticodon is expected to occur with a very low affinity. The observed interaction is some five orders of magnitude stronger than that calculated for duplex formation between two trinucleotides, yet only one tenth the affinity observed for the interaction of the anticodon of native E. coli tRNA^{Glu} with the complementary anticodon of yeast tRNA^{Phe}.

It has been reported that the Specifier Loop glycine codon (nucleotides 99-101) was protected from Mg²⁺-induced cleavage in vitro when tRNA^{Gly}_{GCC} was present (M R Yousef et al., (2005) J. Mol. Biol. 349:273-287). In contrast to A₉₈, the conserved purine A₁₀₂, 3′ to the codon, was protected from Mg²⁺ cleavage in the presence of tRNA. This suggests that pairing of the position 102 purine of the 5′-UTR with the invariant U₃₃ of the tRNA may facilitate the Specifier Loop-tRNA interaction. Thus, in our experimental model of the GlyRS mRNA 5′-UTR, A₁₀₂ may bind to the ASL^{Gly}_{GCC} U₃₃ forming the tetranucleotide duplex:

Specifier Loop: 2AP₉₈GGCA₁₀₂
●●●◯
Anticodon Loop: CCGU₃₃

In binding ASL^{Gly}_{GCC} to the 5′-UTR, we chose to monitor the fluorescence of 2AP₉₈ to avoid interference with the U₃₃-A₁₀₂ interaction. The postulated tetranucleotide duplex may be a contributing factor to the significant binding affinity observed in our fluorescence assays.

The interaction of the glyQS 5′-UTR with tRNA^{Gly} was evident in experiments conducted with as little as 5 mM Mg²⁺ (M R Yousef et al., (2005) J. Mol. Biol. 349:273-287). With our experimental model RNAs in which codon/anticodon interaction was monitored with fluorescence spectroscopy, Mg²⁺ was not required. Gel mobility shift and fluorescence studies indicated that the Specifier/Common complex reconstituting the 5′-UTR had a significant binding affinity (K_{d}s=10.5 μM). The interaction of ASL^{Gly}_{GCC} with the Specifier/Common complex did not generate a supershifted complex in gel electrophoresis. The failure to observe a supershift could be due to lower stability of the ASL-Stem I interaction (K_{d} of 20.2 μM), kinetic on/off rates, or may be a consequence of the electrophoresis method.

In order to confirm the specificity of codon-anticodon interaction within the experimental model, the Specifier codon was changed to that of valine. The new Stem I construct now bound ASL^{Val}, but not ASL^{Arg}, substantiating the availability of the codon and the selectivity of the system. The only published mutations in Stem I were studies of the RNA structural motif called a K-turn (W C Winkler, et al. (2001) RNA 7:1165-1172). The K-turn is composed of the dinucleotide GA on both sides of a loop, such as the two highly conserved GA sequences at the bottom of the 5′-UTR Stem I (FIGS. 1 and 2). A single site mutation in the GA sequence resulted in a significant decrease in expression of the B. subtilis tyrS gene in vivo. However, ASL was still able to bind the Specifier when the same single nucleoside substitution was made in vitro. Since the effect of the K-turn motif has not been tested in the context of the glyQS gene, subsequent experiments are required both in vivo and in vitro to determine the full capacity of the experimental model to mimic the in vivo results.

RNA:RNA biophysical and chemical interactions are composed of the four chemistries of adenosine, uridine, guanosine and cytidine, contrasting with, and probably pre-dating, the 20 chemistries of a protein's amino acids. The unique interaction of the 5′-UTR of aaRS mRNAs with unacylated tRNA in Gram-positive organisms, without a requirement for ribosomes or protein factors, may represent an early mechanism in the regulation of transcription, similar to the binding of small molecules by riboswitch RNAs. Our experimental model supports the proposition that regulation occurs in the absence of protein and is dependent on tRNA's anticodon interaction with codon in the 5′-UTR of nascent mRNA. The Specifier Sequence/tRNA anticodon interaction is very similar to the decoding of mRNA that occurs on the ribosome between the mRNA and tRNA with the exception of the possible binding of conserved A₁₀₂ to tRNA's invariant U₃₃. Participation of U₃₃ in ribosomal codon binding would result in a translational frame shift. However, a tetranucleotide duplex formation between the Specifier Loop and the tRNA's anticodon loop is similar to the tetranucleotide duplex formed between the UGGN sequence in the antiterminator bulge and the tRNA's universal 3′-terminal CCA sequence plus the 5′-adjacent discriminator base in the tRNA.

Some modifications in the anticodon region of tRNAs are essential for the tRNA to form the correct tertiary structure and to function in productively binding to the codon in the ribosomal A-site. The modifications essential for translation may also be critical for transcription regulation in Gram-positive organisms. The GlyRS/tRNA^{Gly} system has no requirement for tRNA modification, but other systems may. The model system that we designed has many of the properties of the T box transcription regulation system studied both in vivo and in vitro. The simplified character of the model permits investigation of the chemically and physically important aspects of 5′-UTR folding and its interactions with tRNA.

High Throughput Analysis. The method disclosed herein for screening a compound for the ability to interfere with binding of a tRNA molecule by an mRNA molecule can also be conducted as a high throughput assay. High throughput analysis can be done with combinatorial libraries to select compounds that interfere with the binding of tRNA to nascent mRNA. Exemplary combinatorial libraries are those containing small compounds for large scale screening analysis. The assay can be done in a microtiter plate format allowing small molecule interference to be observed in a high throughput fashion. The microtiter plate could have 6, 24, 96, 384 or even 1536 sample wells. Methods for handling of microtiter plates and for high throughput analysis of reporter assays are well known in the art and include those using plate readers and/or robots. Alternative alterations of the nascent mRNA may be useful for high throughput analysis and include attaching biotin to one end for covalent attachment to strepavidin-coated microplates.

Except as otherwise indicated, standard methods known to those skilled in the art may be used for the construction and use of nucleic acid molecules and reporter molecules. Such techniques are known to those skilled in the art. See, e.g., Current Protocols In Molecular Biology, edited by F. M. Ausubel et al. (John Wiley & Sons, Inc.; Hoboken, N.J.).

All publications, patents, and patent publications cited herein are incorporated by reference in their entireties for the teachings relevant to the sentence and/or paragraph in which the reference is presented unless otherwise indicated.

That which is claimed is:

1. A method for identifying a compound that interferes with the binding of a tRNA molecule comprising an anticodon to a nascent mRNA molecule comprising a complementary codon in the absence of protein, comprising:
(a) contacting the compound with an RNA reporter construct comprising (i) a reporter molecule and (ii) an RNA molecule comprising the complementary codon and representing a 5′ UTR of the nascent mRNA molecule; and
(b) measuring the binding of the RNA reporter construct and the tRNA molecule in the presence of the compound,
wherein a decrease of binding identifies a compound that interferes with the binding of the tRNA molecule comprising the anticodon to the nascent mRNA molecule comprising the complementary codon.

2. The method of claim 1, wherein said 5′ UTR contains a T box transcription termination control system.

3. The method of claim 2, wherein said 5′ UTR belongs to a gene for an amino acid metabolic enzyme.

4. The method of claim 3, wherein said amino acid metabolic enzyme is a gram positive bacteria amino acid metabolic enzyme.

5. The method of claim 4, wherein said gram positive bacteria amino acid metabolic enzyme is selected from the group consisting of aminoacyl-tRNA synthetases, amino acid biosynthetic enzymes, and amino acid transporter proteins.

6. The method of claim 1, said reporter construct consisting essentially of: (i) a reporter molecule and (ii) an RNA molecule comprising the complementary codon and representing a 5′ UTR of the nascent mRNA molecule, wherein said RNA molecule is not more than 100 nucleotides in length.

7. The method of claim 1, wherein said compound is a member of a compound library.

8. The method of claim 1, wherein said compound is a candidate anti-infective therapeutic and/or disinfecting agent for Gram-positive bacteria.

9. The method of claim 1, wherein:
said 5′ UTR contains a T box transcription termination control system that belongs to a gene for a gram negative bacteria amino acid metabolic enzyme selected from the group consisting of aminoacyl-tRNA synthetases, amino acid biosynthetic enzymes, and amino acid transporter proteins; and
said reporter construct consists essentially of: (i) a reporter molecule and (ii) an RNA molecule comprising the complementary codon and representing a 5′ UTR of the nascent mRNA molecule, wherein said RNA molecule is not more than 100 nucleotides in length.

10. An anti-infective and/or disinfecting compound for Gram-positive bacteria identified by the method of claim 1.

11. An RNA reporter construct useful for measuring the binding of a tRNA molecule to a nascent mRNA molecule of a target gene, comprising:
(a) a reporter molecule; and
(b) an RNA molecule comprising a complementary codon and representing the 5′ UTR of the nascent mRNA molecule of the target gene.

12. The construct of claim 11, wherein said target gene is a gene having a T box transcription termination control system.

13. The construct of claim 11, wherein said construct is unimolecular.

14. The construct of claim 11, wherein said construct comprises at least two molecules.

15. The construct of claim 11, wherein said reporter molecule is a fluorescent reporter molecule.

16. The construct of claim 15, wherein said fluorescent reporter molecule is 2-aminopurine ribonucleoside.

17. The construct of claim 11, wherein said 5′ UTR contains at least the specifier segment of a T box transcription termination control system.

18. The construct of claim 11, wherein said 5′ UTR belongs to a gene for an amino acid metabolic enzyme.

19. The construct of claim 18, wherein said amino acid metabolic enzyme is a gram positive bacteria amino acid metabolic enzyme.

20. The construct of claim 19, wherein said gram positive bacteria amino acid metabolic enzyme is selected from the group consisting of aminoacyl-tRNA synthetases, amino acid biosynthetic enzymes, and amino acid transporter proteins.

21. The construct of claim 11, said reporter construct consisting essentially of: (i) a reporter molecule and (ii) an RNA molecule comprising the complementary codon and representing a 5′ UTR of the nascent mRNA molecule, wherein said RNA molecule is not more than 100 nucleotides in length.
